(19) United States
(12) Patent Application Publication
(10) Pub. No.: US 2007/0166244 A1
(43) Pub. Date: Jul. 19, 2007

(54) Compositions comprising silicone pressure sensitive adhesives for delivering oral care substances

(75) Inventors: Chanchal Kumar Ghosh, West Chester, OH (US); Stephen Andras Kovacs, Loveland, OH (US); Satyanarayana Majeti, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/334,928

(22) Filed: Jan. 19, 2006

(51) Int. Cl. A61K 8/89

(52) U.S. Cl. 424/49

(57) ABSTRACT

Disclosed are compositions for delivering one or more oral care substances to the oral cavity, comprising: (a) a silicone pressure sensitive adhesive selected from a silicone/resin copolymer with silicon-bonded hydroxyl radicals, a silicone/resin copolymer with endcapped silicon-bonded hydroxyl radicals and mixtures thereof; (b) a plasticizing material; (c) a water-soluble bioadhesive polymer; (d) a hydrophilic surfactant, and (e) at least one oral care substance. The silicone/resin copolymer is prepared by polycondensing a silanol endblocked polydialkylsiloxane and a hydroxyl endblocked silicate resin. The polycondensation product may be further reacted with a trialkylsilyl endcapping agent to reduce the number of hydroxyl radicals in the polymer. Further disclosed are methods of using these compositions, such as for teeth bleaching and whitening applications.

### FIELD OF THE INVENTION

The present invention relates to compositions for delivering oral care substances to the oral surfaces such as teeth. The composition forms an adherent film on the surface to which it has been applied and provides sustained release of the oral care substance from the film for prolonged therapeutic, prophylactic, and/or cosmetic benefits.

### BACKGROUND OF THE INVENTION

Oral care products by which various oral care substances or actives can be delivered to the soft and hard tissues of the oral cavity have previously been disclosed. Examples of such oral care products include, for example, brushing aids such as dentifrice products for delivery of anti-caries actives such as fluoride or other actives for the reduction of the bacteria that lead to the formation of plaque, and mouthwashes containing breath freshening actives and/or anti-bacterial actives. In addition, bleaching agents such as peroxide that can be applied directly to the surfaces of the teeth, i.e., to the tooth enamel, have been developed.

However, it has been found that such conventional product forms typically do not provide sufficient substantivity to maintain actives on the hard and soft oral tissues for a period of time sufficient to enhance or prolong the therapeutic, prophylactic, and/or cosmetic benefits provided by the actives. Neither have such conventional product forms been able to provide sustained delivery of oral care actives, without periodic reapplication at relatively short time intervals, or without a special delivery device or containment means such as a mouthpiece.

Attempts have previously been made to enhance the substantivity of whitening bleaches, bactericides, and other active components of oral care products. See, e.g., U.S. Pat. No. 5,425,953 to Sintov et al.; U.S. Pat. No. 5,438,076 to Friedman et al. and U.S. Pat. No. 6,083,421 to Huang. These disclosures focus on water-soluble systems, which are readily dissolved by saliva, generally within about 1-3 hours after application. Therefore, their degree of durability is low, and they do not provide long-term delivery of the active ingredient that is present in the composition. In addition, their water-soluble nature precludes them from being used with oral care actives that would be unstable in water-based films. Sodium percarbonate is one example of such an active; it would be unstable in the high pH environment of an aqueous-based film.

In order to provide an applied composition with a relatively higher degree of durability, the use of water-insoluble or protective coatings that are applied to the teeth has been described. See e.g., U.S. Pat. Nos. 6,589,512; 6,569,408; 6,692,727; 6,649,147; and 6,685,921 all commonly assigned, which disclose compositions containing particular crosslinked organosiloxane resins as the principal delivery agent providing substantivity or adherence to teeth and other oral surfaces. U.S. Pat. No. 5,401,528, to Schmidt discloses organically modified silicic acid polycondensates which are deposited on the teeth, then polymerized in-situ by curing, to coat the teeth in order to protect them from plaque deposits. This system is not a true delivery system by which an active ingredient is released over time; instead, it provides a barrier by which the deleterious effect of plaque-causing bacteria may be diminished. Although such a barrier coating may offer a benefit in terms of enhanced durability, it requires the use of special equipment and complex application; thus, it cannot be performed at home and cannot be used for self-treatment.

There remains a continuing need for convenient delivery systems for various oral care actives in which the substantivity as well as release profile of the active ingredients to teeth and other oral surfaces is enhanced, thereby increasing the efficacy of the treatment.

### SUMMARY OF THE INVENTION

The present invention is directed to compositions and their use for delivering an oral care substance to the oral cavity, comprising:

(a) a silicone pressure sensitive adhesive selected from a silicone/resin copolymer with silicon-bonded hydroxyl radicals, a silicone/resin copolymer with endcapped silicon-bonded hydroxyl radicals and mixtures thereof;
(b) a plasticizing material;
(c) a water-soluble bioadhesive polymer;
(d) a hydrophilic surfactant, and
(e) at least one oral care substance.
   The silicone/resin copolymer is prepared by polycondensing a silanol endblocked polydialkylsiloxane and a hydroxyl endblocked silicate resin. The polycondensation product may be further reacted with a trialkylsilyl endcapping agent to reduce the number of hydroxyl radicals in the polymer.

These and other features, aspects, and advantages of the invention will become evident to those skilled in the art from a reading of the present disclosure.

### DETAILED DESCRIPTION OF THE INVENTION

While the specification concludes with claims particularly pointing out and distinctly claiming the invention, it is believed that the present invention will be better understood from the following description.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

Herein, “comprising” means that other steps and other components which do not affect the end result can be added. This term encompasses the terms “consisting of” and “consisting essentially of.”

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

By “oral composition” is meant a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral composition of the present invention may be in various forms including toothpaste, dentifrice, tooth gel, subgingival gel, mouthrinse, denture product, mouthspray, lozenge, chewable tablet or chewing gum. The oral composition may also be incorporated onto strips or films for direct application or attachment to oral surfaces.

The term “dentifrice”, as used herein, means paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having the gel surrounding the paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices.

The term “teeth”, as used herein, refers to natural teeth as well as artificial teeth or dental prosthesis.

The term “orally acceptable carrier” as used herein includes any safe and effective materials for use in the compositions of the present invention. Such materials conventional additives in oral care compositions including but not limited to fluoride ion sources, anti-calculus or anti-tartar agents, buffers, abrasives such as silica, peroxide sources, alkali metal bicarbonate salts, thickening materials, humectants, water, surfactants, titanium dioxide, flavor system, sweetening agents, xylitol, coloring agents, and mixtures thereof.

The present invention provides compositions which function as an effective delivery matrix for oral care actives in that they provide adhesion and retention of the composition on oral surfaces such as teeth, as well as an effective release profile of such actives on to the surface being treated. The oral compositions thus comprise in addition to the oral care active(s), a combination of materials that provide (1) storage stability of the active(s), (2) adhesion to dry or wet oral surfaces, (3) effective release of actives to the target oral surface(s) and (4) retention on the target surface for a sufficient period of time to achieve the desired effect(s) from the active(s). The essential and optional components of the present compositions are described below.

Silicone Pressure Sensitive Adhesive

The present compositions comprise a silicone pressure sensitive adhesive (PSA) to provide sufficient adhesion to and retention on teeth and other oral surfaces. By “sufficient adhesion” is intended to mean that the composition easily adheres to the target surface after application without requiring an undue amount of pressure to get the composition to adhere to the surface and be maintained in contact thereon. Suitable silicone PSA's include the polycondensation product between a silanol endblocked polydiorganosiloxane and a hydroxyl endblocked silicate resin and encapped versions of such polycondensation product. The polycondensation product is a silicone/resin copolymer with silicon-bonded hydroxyl radicals. Reacting the copolymer with endcapping reagents results in reducing the number of hydroxyl radicals in the copolymer.

Silicone pressure sensitive adhesives (PSA) are well known in the art and many are commercially available. Generally, silicone PSA's are produced by either blending or condensing a silanol endblocked polydiorganosiloxane and a hydroxyl endblocked silicate resin. The polycondensation product, referred to as standard silicone PSA, has been found to provide better cohesive properties compared to a simple blend of the components and is thus preferred in the practice of the present invention. Such standard silicone PSA's have been disclosed for example in U.S. Pat. Nos. 2,736,721, 2,814,601, 2,857,356, and 3,528,940. The adhesive properties of such materials can be varied by altering the ratio of units in the starting silicate resin material or the ratio of silicate resin to polydiorganosiloxane. Examples with optimum adhesive properties have a ratio of resin to polydiorganosiloxane in the range of 40:60 to 65:35. Silicone pressure-sensitive adhesives are known to be non-irritating and non-sensitizing to the skin and have been used as adhesive layers in transdermal drug delivery devices such as those for the controlled release of drugs, such as nitroglycerine. Medical grades of silicone pressure-sensitive are commercially available from Dow Corning Corporation, such as polycondensed PDMS/Resin networks under the BIO-PSA® tradename.

The silicone PSA's may be chemically treated to reduce the content of silicon-bonded hydroxyl radicals, such as described in U.S. Pat. Nos. 4,584,355; 4,585,836 and 4,491,622 all assigned to Dow Corning. This involves reacting the hydroxyl groups with a trialkylsilyl endcapping agent, for example hexamethyldisilazane which results in a trimethylsilyl endcapped silicone PSA. Such encapped silicone PSA's have been labeled “amine compatible” because they exhibit increased chemical stability in the presence of amines compared to the non-encapped or standard silicone PSA's. A preparation scheme for a trimethylsilyl endcapped silicone PSA's is shown below.

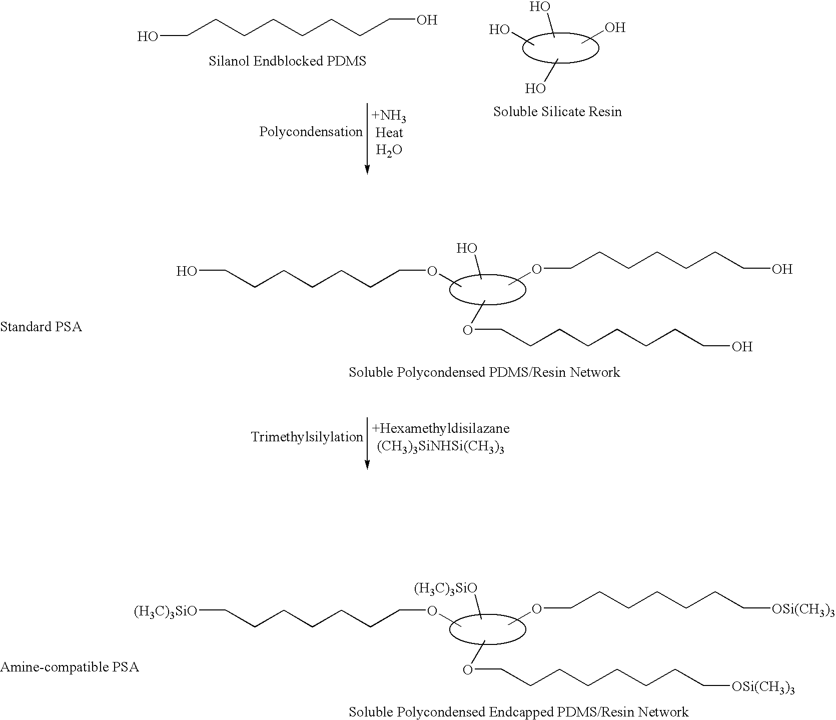

Like standard silicone PSA's, the encapped versions have also been used in transdermal drug delivery devices for the controlled delivery of active pharmaceutical agents amenable to being delivered transdermally for therapeutic purposes such as described in U.S. Pat. Nos. RE 35,474 and 6,337,086. Such encapped silicone PSA's are commercially available from Dow Corning, for example, BIO-PSA® 7-4202 and 7-4302. Particularly useful in the practice of the present invention are BIO-PSA® 7-4202 and 7-4302 polymers having an average molecular weight (AMW) ranging from about 200,000 to about 275,000. These polymers can be solvated in an appropriate solvent such as ethyl acetate, yielding solutions having an average viscosity ranging from about 750 to about 900 centipoise (cp)]

The endcapped silicone PSA may be fully capped or partially capped. In one embodiment the endcapped silicone is at least about 25% capped.

While standard silicone PSA's and endcapped silicone PSA's are useful in the practice of the present invention, the endcapped silicone PSA provides improved stability and compatibility with other components of the matrix by virtue of having none or a reduced number of reactive hydroxyl end groups, while also having the requisite properties for adhesion and retention on the target surface. During endcapping of the silicone PSA, increased crosslinking of the polymer occurs, which creates a relatively stiffer material (increased viscosity) that is tougher to pull off a substrate once it has adhered to it. Thus, the endcapped silicone PSA provides the tack or initial grab to a surface as well as improved durability thereon.

In addition to the adhesion and retention benefits provided by the silicone PSA, a surprising benefit is anti-sensitivity. It is believed that the silicone PSA that adheres to teeth also effectively occludes or blocks dentinal tubule orifices, thereby reducing fluid movement in the tubules and thus, reducing sensitivity for example to cold liquids.

The level of silicone pressure sensitive adhesive resin that is used in the compositions is dependent on a number of factors including its degree of solubility or miscibility in the formulation. Generally, the range of silicone PSA used in the present invention ranges from about 1% to about 70%. In dentifrices, the level typically ranges from about 1% to about 10%. In embodiments such as gels for direct application to teeth by painting on or incorporated onto strips or films for attachment to oral surfaces, higher levels are typically used, ranging from about 10% to about 70%, from about 20% to about 60% in certain embodiments.

Plasticizing Material

In addition to the silicone pressure sensitive adhesive, the delivery matrix of the present invention further comprises one or a mixture of plasticizing materials to control both the adhesive and cohesive properties of the matrix. The plasticizing agent softens the silicone PSA, to allow adhesion particularly to wet surfaces such as teeth. The plasticizing agent may also function to maintain the viscosity of the matrix at a high enough level to prevent other components especially actives from precipitating out of the matrix. As plasticizing material, fluid diorganopolysiloxane-based polymers have been found useful. The fluid diorganosiloxane polymers span a large range of viscosities, from about 10 to about 1,000,000 centistokes (cSt) at 25° C. Among the fluid diorganopolysiloxane-based polymers of the present invention are diorganopolysiloxane polymers comprising repeating units corresponding to the formula (R₂SiO)ₙ, where R is a monovalent radical containing from 1 to 6 carbon atoms, preferably selected from the group consisting of methyl, ethyl, propyl, isopropyl, butyl, isobutyl, t-butyl, amyl, hexyl, vinyl, allyl, cyclohexyl, amino alkyl, phenyl, fluoroalkyl and mixtures thereof. The fluid diorganopoylsiloxane polymers employed in the present invention may contain one or more of these radicals as substituents on the siloxane polymer backbone. The fluid diorganopolysiloxane polymers may be terminated by triorganosilyl groups of the formula (R′₃Si) where R′ is a monovalent radical selected from the group consisting of radicals containing from 1-6 carbon atoms, hydroxyl groups, alkoxyl groups and mixtures thereof. The fluid diorganopolysiloxane polymer is miscible with the silicone pressure sensitive adhesive and other components in ratios required for a specific formulation. For example, a mixture of a relatively low viscosity fluid diorganopolysiloxane polymer (viscosity ranging from about 10 to about 12,500 cSt.) and a higher viscosity polymer (viscosity ranging from about 12,500 to about 100,000 cSt) has been found particularly useful. The low viscosity fluid diorganopolysiloxane polymer functions mainly to soften the silicone PSA and the higher viscosity fluid diorganopolysiloxane polymer additionally aids in maintaining a high viscosity to suspend a solid component such as carbamide peroxide and to prevent it from precipitating out of the matrix. In one embodiment, the plasticizing material comprises a mixture of fluid polydimethylsiloxane (PDMS) polymers such as supplied by Dow Corning under the tradenames DC Q7-9120 (about 100 cSt) and DC 200 Fluid (about 60,000 cSt). Fluid diorganopolysiloxane polymers such as these are also available from the General Electric Company and from Wacker Silicones.

The plasticizing material is included in an amount sufficient to soften the silicone PSA. The ratio of silicone PSA resin to fluid diorganopolysiloxane-based polymer can vary widely depending on the product form. For dentifrices, the ratio may be from to about 2:1 to about 1:40. For gels to be directly applied to an oral surface, the ratio may range from about 10:1 to about 1:10, typically from about 6:1 to about 1:1.

Bioadhesive Material

A third component of the present compositions is a bioadhesive material which functions to enhance the adhesive property of the silicone PSA in a wet environment over a longer period of time. Suitable bioadhesive materials are hydrophilic in character and provide many other desirable properties to the matrix such as binding, thickening, and film formation. It is believed the bioadhesive material allows water into the substantially anhydrous and hydrophobic silicone matrix thereby facilitating release of actives, particularly those that are water soluble, from the silicone matrix to the target wet surface, such as teeth. Useful bioadhesive materials include polyhydric alcohols such as sorbitol and glycerin and derivatives thereof such as ethers and esters; polymers or copolymers of ethylene oxide, propylene oxide, acrylates and vinylpyrrolidone and hydroxyethylcellulose polymers. Examples include commercially available materials such as polyethylene oxide under the tradename Polyox from Dow Chemical Company; block copolymers of ethylene oxide and propylene oxide designated under the tradenames Pluronic and Pluraflo from BASF, crosslinked acrylate polymers designated under the trademark Carbopol and the Pemulen® series available from Noveon Incorporated; sorbitol powder or 70% sorbitol solution available from Lipo Chemicals and from Roquette America; triacetin (triester of glycerin and acetic acid) available from Eastman Chemical; and polyvinylpyrrolidone available from ISP under the tradename Plasdone.

The bioadhesive polymer is generally present in the composition at a level ranging from about 0.5% to about 50% by weight.

Hydrophilic Surfactant

Another component of the present compositions is a hydrophilic surfactant which functions to promote migration and release of actives, such as peroxide, from the substantially hydrophobic silicone adhesive matrix to wet surfaces such as teeth. The hydrophilic surfactant allows water into the matrix, which initiates the release of the active. As active is released, channels are created in the matrix that allow more water, which then allow further release of active. For example, when an active such as a peroxide compound is incorporated in the present delivery matrix, an optimum release profile is achieved. Increasing amounts of peroxide are released over a period of time for a sustained bleaching effect.

Suitable hydrophilic surfactants include nonionic surfactants, which are water soluble, low-foaming and have a hydrophilic/lipophilic balance (HLB) number ranging from about 3 to about 15. Examples include surfactants based on glycols and alkylene oxides, such as ethylene glycol, propylene glycol, ethylene oxide and propylene oxide. Particularly useful nonionic surfactants include polymers and copolymers of ethylene and propylene oxide, ethoxylated and/or propoxylated fatty acids, alcohols or alkylphenols and silicone polyethers. Such surfactants are commercially available as detailed in 2005 McCutcheon's, vol. 1 Emulsifiers & Detergents. Examples include ethoxylated alcohols or alkylphenol such as supplied by Uniqema under the tradename Synperonic and by BASF under the tradenames Lutensol and Plurafac; block copolymers of ethylene oxide and propylene oxide such as supplied by Sanyo Chemical under the tradename Newpol and by BASF under Pluronic L tradename; and silicone polyethers, such as supplied by General Electric under the tradename Silwet and by Goldschmidt under the Abil EM tradename. Similar silicone polyether surfactants are available from Dow Coming, such as sold under the designation DC 193 Fluid and from OSI Specialties under the tradename Silsoft (polyethylene glycol derivative of dimethicone).

The hydrophilic surfactant is generally present in an amount ranging from about 0.5% to 10% by weight.

Oral Care Substances

The delivery matrix prepared from the combination of the above described components is useful to deliver oral care substances to wet oral surfaces, especially teeth and gums.

The oral care substance preferably contains an active at a level where upon directed use, the benefit sought by the user is promoted without detriment to the oral surface to which it is applied. Examples of benefits these actives address include, but, are not limited to, appearance and structural changes to teeth including whitening, stain bleaching, stain removal, prevention and treatment of plaque, tartar, caries, cavities and dentinal sensitivity; treatment of oral cavity conditions such as inflamed and/or bleeding gums, mucosal wounds, lesions, ulcers, aphthous ulcers, cold sores, and tooth abscesses; and elimination of mouth malodor resulting from the conditions above and other causes such as microbial proliferation.

Suitable oral care substances include any material that is generally considered safe for use in the oral cavity and that provides changes to the overall appearance and/or health of the oral cavity. The level of oral care substance in the compositions of the present invention is generally, unless specifically noted, from about 0.01% to about 50%. Depending upon the type of active and the condition being treated, the level of active may be from about 0.1% to about 20%, or from about 0.5% to about 10%, or from about 1% to about 7%, by weight of the composition.

Oral care compositions or substances of the present invention may include many of the actives previously disclosed in the art. The following is a non-limiting list of oral care actives that may be used in the present invention.

1. Teeth Whitening Actives

Teeth whitening actives may be included in the oral care substance of the present invention. The actives suitable for whitening are selected from the group consisting of the peroxides, metal chlorites, perborates, percarbonates, peroxyacids, persulfates, and combinations thereof. Suitable peroxide compounds include hydrogen peroxide, urea peroxide, calcium peroxide, and mixtures thereof. Suitable metal chlorites include calcium chlorite, barium chlorite, magnesium chlorite, lithium chlorite, sodium chlorite, and potassium chlorite. Additional whitening actives may be hypochlorite and chlorine dioxide. A preferred percarbonate is sodium percarbonate. Preferred persulfates are oxones.

2. Anti-Tartar Agents

Anti-tartar agents known for use in dental care products include phosphates. Phosphates include pyrophosphates, polyphosphates, polyphosphonates and mixtures thereof. Pyrophosphates are among the best known for use in dental care products. Pyrophosphate and polyphosphate ions are delivered to the teeth derive from pyrophosphate or polyphosphate salts. The pyrophosphate salts useful in the present compositions include the dialkali metal pyrophosphate salts, tetra-alkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. While any of the above mentioned pyrophosphate salts may be used, tetrasodium pyrophosphate salt is preferred. Sodium polyphosphate and triethanolamine polyphosphates, for example, are also useful.

The pyrophosphate salts are described in more detail in Kirk & Othmer, Encyclopedia of Chemical Technology, Third Edition, Volume 17, Wiley-Interscience Publishers (1982). Additional anticalculus agents include pyrophosphates or polyphosphates disclosed in U.S. Pat. No. 4,590,066 issued May 20, 1986; polyacrylates and other polycarboxylates such as those disclosed in U.S. Pat. No. 3,429,963 issued Feb. 25, 1969 and U.S. Pat. No. 4,304,766 issued Dec. 8, 1981; and U.S. Pat. No. 4,661,341 issued Apr. 28, 1987; polyepoxysuccinates such as those disclosed in U.S. Pat. No. 4,846,650 issued Jul. 11, 1989; ethylenediaminetetraacetic acid as disclosed in British Patent No. 490,384 dated Feb. 15, 1937; nitrilotriacetic acid and related compounds as disclosed in U.S. Pat. No. 3,678,154 issued Jul. 18, 1972; polyphosphonates as disclosed in U.S. Pat. No. 3,737,533 issued Jun. 5, 1973, U.S. Pat. No. 3,988,443 issued Oct. 26, 1976 and U.S. Pat. No. 4,877,603 issued Oct. 31, 1989.

Other anticalculus agents that may be used in place of or in combination with the pyrophosphate salt include such known materials as synthetic anionic polymers including polyacrylates and copolymers of maleic anhydride or acid and methyl vinyl ether (e.g., Gantrez), as described, for example, in U.S. Pat. No. 4,627,977; as well as, e.g., polyamino propane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

3. Fluoride Ion Source

Fluoride ion sources are well known for use in oral care compositions as anticaries agents. Fluoride ions are contained in a number of oral care compositions for this purpose, particularly toothpastes. Patents disclosing such toothpastes include U.S. Pat. Nos. 3,538,230, Nov. 3, 1970; 3,689,637, Sep. 5, 1972; 3,711,604, Jan. 16, 1973; 3,911,104, Oct. 7, 1975; 3,935,306, Jan. 27, 1976; and 4,040,858, Aug. 9, 1977.

Application of fluoride ions to dental enamel serves to protect teeth against decay. A wide variety of fluoride ion-yielding materials can be employed as sources of soluble fluoride in the instant compositions. Examples of suitable fluoride ion-yielding materials are found in U.S. Pat. No. 3,535,421; issued Oct. 20, 1970 and U.S. Pat. No. 3,678,154; issued Jul. 18, 1972, such as sodium fluoride, potassium fluoride, stannous fluoride and ammonium fluoride. In one embodiment, the instant compositions provide from about 50 ppm to 10,000 ppm; in another embodiment from about 100 to 3000 ppm, of fluoride ions in the compositions that contact dental surfaces when used with the delivery system of the present invention.

4. Anti-Microbial Agents

Anti-microbial agents can also be present in the oral care compositions or substances of the present invention. Such agents may include, but are not limited to, 5-chloro-2-(2,4-dichlorophenoxy)-phenol, commonly referred to as triclosan, and described in The Merck Index, 11 th ed. (1989), pp. 1529 (entry no. 9573) in U.S. Pat. No. 3,506,720, and in European Patent Application No. 0,251,591, published Jan. 7, 1988; phthalic acid and its salts including, but not limited to those disclosed in U.S. Pat. No. 4,994,262, Feb. 19, 1991, preferably magnesium monopotassium phthalate, chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024); tetradecylpyridinium chloride (TPC); N-tetradecyl-4-ethylpyridinium chloride (TDEPC); octenidine; delmopinol, octapinol, and other piperidino derivatives; nicin preparations; zinc/stannous/copper ion agents; antibiotics such as augmentin, amoxicillin, tetracycline, doxycycline, minocycline, and metronidazole; and analogs and salts of the above; essential oils including thymol, geraniol, carvacrol, citral, hinokitiol, eucalyptol, catechol (particularly 4-allyl catechol) and mixtures thereof; methyl salicylate; hydrogen peroxide; metal salts of chlorite and mixtures of the above.

5. Anti-Inflammatory Agents

Anti-inflammatory agents can also be present in the oral care compositions or substances of the present invention. Such agents may include, but are not limited to, non-steroidal anti-inflammatory agents or NSAIDs such as ketorolac, flurbiprofen, ibuprofen, naproxen, indomethacin, aspirin, ketoprofen, piroxicam and meclofenamic acid. Use of NSAIDs such as ketorolac are claimed in U.S. Pat. No. 5,626,838, issued May 6, 1997. Disclosed therein are methods of preventing and, or treating primary and reoccurring squamous cell carcinoma of the oral cavity or oropharynx by topical administration to the oral cavity or oropharynx an effective amount of an NSAID.

6. Nutrients

Nutrients may improve the condition of the oral cavity and can be included in the oral care compositions or substances of the present invention. Nutrients include minerals, vitamins, oral nutritional supplements, enteral nutritional supplements, and mixtures thereof.

Minerals that can be included with the compositions of the present invention include calcium, phosphorus, fluoride, zinc, manganese, potassium and mixtures thereof. These minerals are disclosed in Drug Facts and Comparisons (loose leaf drug information service), Wolters Kluer Company, St. Louis, Mo., © 1997, pp 10-17.

Vitamins can be included with minerals or used separately. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Such vitamins are disclosed in Drug Facts and Comparisons, Wolters Kluer Company, © 1997, pp. 3-10.

Oral nutritional supplements include amino acids, lipotropics, fish oil, and mixtures thereof, as disclosed in Drug Facts and Comparisons, Wolters Kluer Company, ©1997, pp. 54-54e. Amino acids include, but, are not limited to L-Tryptophan, L-Lysine, Methionine, Threonine, Levocamitine or L-carnitine and mixtures thereof. Lipotropics include, but, are not limited to choline, inositol, betaine, linoleic acid, linolenic acid, and mixtures thereof. Fish oil contains large amounts of Omega-3 (N-3) Polyunsaturated fatty acids, eicosapentaenoic acid and docosahexaenoic acid.

Enteral nutritional supplements include, but, are not limited to protein products, glucose polymers, corn oil, safflower oil, medium chain triglycerides as disclosed in Drug Facts and Comparisons, Wolters Kluer Company, © 1997, pp. 55-57.

7. Mouth and Throat Products

Other materials that can be used with the present invention include commonly known mouth and throat products. Such products are disclosed in Drug Facts and Comparisons, Wolters Kluer Company, St. Louis, Mo., ©1997, pp. 520b-527. These products include, but, are not limited to anti-fungal, antibiotic and analgesic agents.

8. Antioxidants

Antioxidants are generally recognized as useful in compositions such as those of the present invention. Antioxidants are disclosed in texts such as Cadenas and Packer, The Handbook of Antioxidants, © 1996 by Marcel Dekker, Inc. Antioxidants that may be included in the oral care composition or substance of the present invention include, but are not limited to Vitamin E, ascorbic acid, Uric acid, carotenoids, Vitamin A, flavonoids and polyphenols, herbal antioxidants, melatonin, aminoindoles, lipoic acids and mixtures thereof.

9. H-2 Antagonists

Histamine-2 (H-2 or H2) receptor antagonist compounds (H-2 antagonists) may be used in the oral care composition of the present invention. As used herein, selective H-2 antagonists are compounds that block H-2 receptors, but do not have meaningful activity in blocking histamine-1 (H-1 or H1) receptors. Selective H-2 antagonists stimulates the contraction of smooth muscle from various organs, such as the gut and bronchi; this effect can be suppressed by low concentrations of mepyramine—a typical antihistaminic drug. The pharmacological receptors involved in these mepyramine-sensitive histamine responses have been defined as H-1 receptors (Brit. J. Pharmacol Chemother., Vol. 27 (1966), p. 427). Histamine also stimulates the secretion of acid by the stomach (Proc. Soc. Exp. Biol. Med., Vol. 48 (1941), p. 65), increases the heart rate (J. Pharmacol., Vol. 130 (1960), p. 450), and inhibits contractions in the rat uterus (Brit. J. Pharmacol. Chemother., Vol. 1 (1946), p. 278); these actions cannot be: antagonized by mepyramine and related drugs. The H-2 antagonists useful in the oral care compositions or substances are those that blockade the receptors involved in mepyramine-insensitive, non-H-1 (H-2), histamine responses, and do not blockade the receptors involved in mepyramine-sensitive histamine responses.

Selective H-2 antagonists are those compounds found to be H-2 antagonists through their performance in classical preclinical screening tests for H-2 antagonist function. Selective H-2 antagonists are identified as compounds which can be demonstrated to function as competitive or non-competitive inhibitors of histamine-mediated effects in those screening models specifically dependent upon H-2 receptor function, but to lack significant histamine antagonist activity in those screening models dependent upon H-1 receptor function. Specifically, this includes compounds that would be classified as described by J. W. Black, et al., “Definition and Antagonism of Histamine H2-Receptors”, Nature, Vol. 236 (Apr. 21, 1972), pp. 385-390, as H-2 antagonists if assessed as described by Black through testing with the guinea pig spontaneously beating right atria in vitro assay and the rat gastric acid secretion in vivo assay, but shown to lack in significant H-1 antagonist activity relative to H-2 antagonist activity, if assessed as described by Black with either the guinea pig ileum contraction in vitro assay or the rat stomach muscle contraction in vivo assay. Preferably selective H-2 antagonists demonstrate no significant H-1 activity at reasonable dosage levels in the above H-1 assays. Typical reasonable dosage level is the lowest dosage level at which 90% inhibition of histamine, preferably 99% inhibition of histamine, is achieved in the above H-2 assays.

Selective H-2 antagonists include compounds meeting the above criteria which are disclosed in U.S. Pat. Nos. 5,294,433 and 5,364,616, issued Mar. 15, 1994 and Nov. 15, 1994 respectively and assigned to Procter & Gamble, wherein the selective H-2 antagonist is selected from the group consisting of cimetidine, etintidine, ranitidine, ICIA-5165, tiotidine, ORF-17578, lupitidine, donetidine, famotidine, roxatidine, pifatidine, lamtidine, BL-6548, BMY-25271, zaltidine, nizatidine, mifentidine, BMY-25368 (SKF-94482), BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, and HB-408. Particularly preferred is cimetidine (SKF-92334), N-cyano-N′-methyl-N″-(2-(((5-methyl-1H-imidazol-4-yl)methyl)thio)ethyl)guanidine:

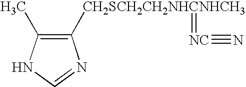

Cimetidine is also disclosed in The Merck Index, 11th edition (1989), p. 354 (entry no. 2279), and Physicians' Desk Reference, 46th edition (1992), p. 2228. Related preferred H-2 antagonists include burimamide and metiamide.

10. Analgesic Actives

Anti-pain or desensitizing agents can also be present in the oral care compositions or substances of the present invention. Such agents may include, but are not limited to, strontium chloride, potassium nitrate, natural herbs such as gall nut, Asarum, Cubebin, Galanga, scutellaria, Liangmianzhen, Baizhi, etc.

11. Anti-Viral Actives

Antiviral actives useful in the present composition include any know actives that are routinely use to treat viral infections. Such anti-viral actives are disclosed in Drug Facts and Comparisons, Wolters Kluer Company, ©1997, pp. 402(a)-407(z). Specific examples include anti-viral actives disclosed in U.S. Pat. No. 5,747,070, issued May 5, 1998. Said Patent discloses the use of stannous salts to control viruses. Stannous salts and other anti-viral actives are described in detail in Kirk & Othmer, Encyclopedia of Chemical Technology, Third Edition, Volume 23, Wiley-Interscience Publishers (1982), pp. 42-71. The stannous salts that may be used in the present invention would include organic stannous carboxylates and inorganic stannous halides. While stannous fluoride may be used, it is typically used only in combination with another stannous halide or one or more stannous carboxylates or another therapeutic agent.

Solvent

A solvent may optionally be present in the present compositions to aid in the miscibility or solvation of various components particularly the silicone pressure sensitive adhesive and the plasticizing agent to form an adhesive and cohesive composition which can be easily applied onto and adhered to teeth or other oral surfaces, for example as a continuous film coating.

The solvent may be a volatile solvent that evaporates from the composition after processing or after application, and comprises from about 1% to about 60% by weight of the composition. Suitable solvents include nontoxic hydrocarbon oils, volatile silicones, non-hydrocarbon solvents, and mixtures thereof.

Hydrocarbon oils useful in the present invention include those having boiling points in the range of 60-260 ° C., such as hydrocarbon oils having from about C₈ to about C₂₀ chain lengths, preferably C₈ to C₂₀ isoparaffins. Examples of useful isoparaffins are isododecane, isohexadecane, isoeicosane, 2,2,4-trimethylpentane, 2,3-dimethylhexane and mixtures thereof. In one embodiment the isoparaffin solvent is isododecane, available for example as, Permethyl 99A from Permethyl Corporation corresponding to the formula: CH₃(CH₂)₁₀CH₃

Volatile silicone fluids include cyclomethicones having 3, 4 and 5 membered ring structures corresponding to the formula: 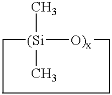
where X is from about 3 to about 6. Such volatile silicones include 244 Fluid, 344 Fluid and 245 Fluid, and 345 Fluid all from Dow Corning Corporation.

The general classes of non-hydrocarbon solvents useful herein include esters, ketones, alcohols, fluorocarbons and fluorocarbon ethers having boiling points in the range of 60 to 200° C. Non-hydrocarbon solvents or mixtures thereof particularly useful include those that are capable of solubilizing the adhesive resin and the plasticizing agent. Such solvents include but are not limited to ethanol, acetone, butanone, ethyl acetate, propyl acetate, amyl acetate, ethyl butyrate, methyl nonafluoroisobutyl ether, methyl nonafluorobutyl ether, and mixtures thereof. These non-hydrocarbon solvents are readily available such as ethyl acetate and methyl ethyl ketone, both supplied by J. T. Baker of Phillispburg, N.J., and HFE (a mixture of methyl nonafluoroisobutyl ether and methyl nonafluorobutyl ether), supplied by the 3M Company.

Rheology Modifiers

The compositions may optionally comprise a rheology modifier which inhibits settling and separation of components or controls settling in a manner which facilitates re-dispersion and may control rheological flow properties. Suitable rheology modifiers herein include organo modified clays, silicas, polyethylene, and mixtures thereof. The preferred organophilic clays comprise quaternium-18 hectorite or Stearalkonium hectorite, such as Bentone 27 and 38™ from Rheox, organoclay dispersion such as Bentone ISD gel™; or bentonite organo modified clays such as Bentone 34™ from Rheox or the Claytone Series™ from Southern Clay Products; and mixtures thereof. The preferred silicas may be fumed silica such as the Aerosil™ series from Degussa or the Cab-o-sil™ M series from Cabot Corporation, silica gels such as the Sylodent™ or Sylox™ series from W. R. Grace & Co. or precipitated silica such as Zeothix 265 from J. M. Huber Corporation.

The rheology modifier may be present in the composition at a level of from about 0.1% to about 30%.

Other Ingredients

In addition to the above materials, a number of other components may desirably be added. Additional components include, but are not limited to, flavoring agents, sweetening agents, xylitol, opacifiers, coloring agents, additional surfactants, and chelants such as ethylenediaminetetraacetic acid. Suitable flavoring agents include, but are not limited to, oil of peppermint, oil of sassafras, clove bud oil, peppermint, menthol, anethole, thymol, methyl salicylate, eucalyptol, cassia, 1-methyl acetate, sage, eugenol, parsley oil, oxanone, oil of wintergreen, alpha-irisone, oil of spearmint, marjoram, lemon, orange, propenyl guaethol, cinnamon, and mixtures thereof.

Pigments may also added to the compositions herein to more precisely indicate the locations at which the composition has actually been applied, allowing the user to apply the composition more thoroughly or evenly.

The present compositions range from substantially non-aqueous to aqueous. By substantially non-aqueous is meant that the compositions may contain very low amounts of water, less than about 5%, which is typically introduced in the composition with other materials, such as with sorbitol or other hygroscopic materials. Water employed in the preparation of commercially suitable aqueous compositions should preferably be of low ion content and free of organic impurities. Water generally comprises from about 5% to about 70%, and preferably from about 20% to about 50%, by weight of the aqueous compositions herein. These amounts of water include the free water which is added plus that which is introduced with other materials.

Method of Use

In practicing the present invention, the user need only apply a composition herein that contains the oral care substance or substances necessary in order to obtain a desired effect, e.g., whitening, breath freshening, caries prevention, pain relief, desensitizing, gum health, tartar control, etc. to the tooth surfaces in the areas desired. The compositions may also be applied to other surfaces of the oral cavity, such as the gingival or mucosal tissues, or to any other oral cavity surface. The composition can be applied with a brush, a pen applicator, a doe's foot applicator, or the like, or even with the fingers. The oral composition may also be incorporated onto strips or films for direct application or attachment to oral surfaces. Examples of suitable strips which are flexible and conformable are described for example in commonly assigned U.S. Pat. Nos. 5,879,691; 5,891,453; 5,894,017; 5,989,569; 6,045,811; 6,096,328; and 6,136,297.

A film containing the oral care substance quickly forms on the surface to which the composition has been applied. Prolonged delivery of the oral care substance is made possible as the oral care substance is released from the film over time. Then, any residual product may be easily removed by wiping, brushing or rinsing the oral surface after a desired period of time has elapsed, or in the normal course of tooth brushing or other oral care activities. Preferably, the compositions are almost unnoticeable when applied to the oral cavity.

It is not necessary to prepare the oral cavity before applying the composition of the present invention. For example, the user may or may not choose to brush the teeth or rinse the mouth before applying the composition. The surfaces of the oral cavity are not required to be dried or to be excessively wet with saliva or water before the composition is applied. It is an advantage of the present compositions that adhesion to wet surfaces is improved.

It should be understood that the present invention relates not only to methods for delivering an oral care substance to the oral cavity of a human, but also to methods of delivering an oral care substance to the oral cavity of an animal, e.g., household pets or other domestic animals, or animals kept in captivity.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Example I

### Teeth Whitening Compositions

The present compositions are advantageously used in teeth bleaching or whitening applications. Examples of teeth whitening compositions in accordance with the present invention are fluid compositions that can be applied to the teeth by brushing, by painting onto the tooth enamel surface or by adhering a strip coated with the composition onto teeth. The substantially anhydrous hydrophobic silicone pressure sensitive adhesive of the present invention provides a stable vehicle that prevents the decomposition of the whitening agent such as peroxide during storage and before use. Upon application to the teeth, the applied whitening composition forms an adherent layer of whitening agent-containing product that releases the whitening agent over an extended period of time, e.g., from about 5 minutes to as long as overnight. The applied layer adheres to the tooth surface whereby the released whitening agent then whitens the teeth to which the composition is applied. Peroxide sources are particularly useful as whitening agents.

A series of teeth whitening compositions according to the present invention are shown below with the ingredients in weight %. The whitening compositions are prepared by adding and mixing the ingredients of the composition in a suitable vessel such as a stainless steel tank provided with a mixer to form a homogeneous dispersion or solution.

TABLE 1
Teeth Whitening Compositions

| Component | 1A | 1B | 1C | 1D | 1E | 1F | 1G | 1H | 1J | IK |
|---|---|---|---|---|---|---|---|---|---|---|
| Silicone PSA¹ | 41.42 | 48.42 | 48.92 | 59.35 | 48.92 | 48.42 | 45.92 | 53.92 | 50.62 | 47.50 |
| Peroxide Source² | 28.58 | 28.58 | 28.58 | 17.15 | 28.58 | 28.58 | 28.58 | 28.58 | 28.58 | 28.58 |
| PDMS (60,000 cst)³ | 4.70 | 4.70 | 4.70 | 4.70 | 4.70 | 4.70 | 4.70 | 3.70 | 4.70 | 4.82 |
| PDMS (100 cst)⁴ | 10.30 | 10.30 | 10.30 | 10.30 | 10.30 | 10.30 | 12.30 | 8.30 | 10.30 | 10.00 |
| Silicone Polyether⁵ | 5.00 | 5.00 | 5.00 | 5.00 | 5.00 | 5.00 | 5.00 | 3.00 | 2.50 | 5.00 |
| Polyethyleneoxide⁶ | 10.00 | | | | | | | | | |
| Polyacrylate⁷ | | 2.50 | 2.50 | 2.50 | | | 3.50 | 2.50 | 2.50 | 3.00 |
| Sorbitol | | | | | 2.50 | | | | | |
| Triacetin | | | | | | 2.50 | | | | |
| Sodium Stannate | | | | | | | | | 0.30 | 0.60 |
| Flavor | | 0.50 | | 1.00 | | 0.50 | | | 0.50 | 0.50 |

¹BIO-PSA ® 7-4202 or 7-4302 amine compatible adhesive resin from Dow Corning.
²Urea hydrogen peroxide (35% H₂O₂) or sodium percarbonate
³Dow Corning 200 Fluid (polydimethylsiloxane)
⁴Dow Corning Q7-9120 Fluid (polydimethylsiloxane)
⁵Dow Corning DC193 Fluid, Silwet L from GE or Silsoft (430, 440, 475, 840) from OSI Specialties
⁶Dow Polyox WSR 301
⁷Pemulen TR-2 (Acrylate Crosspolymer) or Carbopol from Noveon

The embodiments disclosed and represented by the teeth whitening compositions above have many advantages. For example, they provide better durability and sustained delivery of bleaching agent particularly to the surfaces of the teeth. They also provide a convenient, discrete, and easy to use product form which can deliver benefits that are significantly different from those that can be achieved by conventional product forms.

Other oral care actives may be used in addition to or instead of the bleaching agent in similar compositions, for example, sodium or stannous fluoride, sodium monofluorophosphate, pyrophosphate, chlorhexidine, polyphosphate, triclosan, enzymes and flavors, to provide additional benefits in addition to whitening, stain bleaching and stain removal. These benefits include, but are not necessarily limited to: fluoridation and remineralization, plaque and tartar removal and prevention. Flavors would enhance consumer acceptability of the treatment compositions. In addition, oral care actives that would exhibit instability in an aqueous-based film system can be incorporated into the substantially anhydrous compositions herein without compromising stability.

### Example II

### Bleaching Performance

The bleaching performance of compositions against intrinsic tooth stains may be evaluated using the following procedure. Extracted human molars are cleaned of any soft tissue, and polished/prophied to remove any tartar or extrinsic stains. The molar specimens are mounted into Lego ®0 blocks baseline and CIE L*a*b* values are measured using a Fuji HC1000 digital camera under controlled lighting conditions (D55 light) with a polarizing filter. The molars are then re-hydrated overnight in either water or phosphate buffer solution. Thereafter, the molars are removed from solution for treatment with the test composition(s). Each test strip is separated from the release liner and wrapped around each molar. A few drops of human saliva were added to each test molar. The molars are then placed in a 37 degree C. incubator during the duration of the treatment. Each molar is treated with the composition for 30 minutes twice daily over the. study period, 4 or more days. After 30 minutes treatment time, the molars are removed from the incubator and rinsed with distilled water to remove any residual composition. The molars are placed in water or buffer solution in between each treatment. Two to four hours are allowed between each treatment period.

After each treatment, the specimens are blotted dry and measured for changes in L*, a*, and b*, a numerical expression of three dimensional color space where L* represents lightness on the y axis, a* represents chroma (red-green) on the x axis, and b* represents chroma (yellow-blue) on the z axis.

The bleaching performance a composition according to the present invention comprising a silicone PSA and 6% hydrogen peroxide was compared to that of a carbopol gel composition containing the same level of hydrogen peroxide (commercial whitening product sold as Crest Whitestrips®), using the above procedure over a 6.5 day period. Test compositions are experimental strips containing about 0.3 g of test composition (Example 1D) compared to Crest Whitestrips® containing about 0.2 g Carbopol gel composition. Condensed results are shown in Table II below as change in delta b* vs. baseline. These results demonstrate that the present matrix containing the silicone PSA provides greater bleaching benefit on intrinsic discolorations/stains of extracted human teeth versus a Carbopol gel containing the same concentration of hydrogen peroxide under aqueous conditions simulating the mouth environment.

TABLE II
Δb* vs. Baseline of Human Enamel

| | Crest Whitestrips | Experimental Strip |
|---|---|---|
| Treatment Time (Hours) | Δb* Change | Δb* Change |
| 0.5 | −0.74 | −0.40 |
| 1.0 | −1.06 | −0.85 |
| 1.5 | −1.40 | −1.35 |
| 2.0 | −1.65 | −1.70 |
| 2.5 | −1.88 | −2.15 |
| 3.0 | −2.00 | −2.56 |
| 4.0 | −2.25 | −3.00 |
| 5.0 | −2.55 | −3.30 |
| 5.5 | −2.73 | −3.40 |
| 6.5 | −3.01 | −3.50 |

### Example III

### Release of Bleaching Agent (Peroxide) From Silicone PSA Matrix

The effects of the bioadhesive material and surfactant in a silicone PSA matrix on the release of peroxide were studied using the following procedure. The test compositions comprise 40-60% % silicone PSA, 15-20% PDMS as plasticizing material, 6-10% peroxide (added as UHP). The surfactant used was 5% Dow Corning DC193 Fluid.

One liter of de-ionized water (the dissolution medium) was weighed into a glass beaker, the beaker was placed on a lab jack and the jack was adjusted to a height where the impeller blade was level with the 400 mL mark of the beaker. The mixer was then turned on and the speed was set to 100 rpm.

A strip (containing about 0.3 g of the test composition) was selected, separated from the release liner, weighed and laid (gel-side exposed) onto a 1×3 inch unfrosted glass microscope slide. One end of the strip was attached to the slide using a smaller binder clip, while the opposite end of the strip was held in place when the slide was clamped to the stand holding the mixer. After clamping the slide/strip assembly, it was positioned in such a way that the slide was parallel to the beaker wall, and the gel surface was tangential to the rotation of the impeller. The slide was then lowered into the water and monitoring of peroxide released from the strip was started. The peroxide release was monitored by sampling the dissolution medium at selected time points (1 min, 5 min, 10 min, etc) over a period of I hour or longer. The peroxide concentration of each sample was determined using an indicator strip method (RQ Flex reflectometric test).

At the end of the study, the gel composition on the strip was dissolved in ethyl acetate. The dissolved gel was added back into dissolution medium and the resulting mixture was emulsified. The total peroxide content was determined by measuring the peroxide concentration of the emulsion. Table III below shows the % peroxide released from the matrix as a function of time. Results indicate that both surfactant and bioadhesive material are important for controlled and sustained release of peroxide from a silicone PSA matrix.

TABLE III
Peroxide Migration From Silicone PSA Matrix

| | 1 | 5 | 10 | 20 | 30 | 40 | 50 | 60 |
|---|---|---|---|---|---|---|---|---|
| Formula Type | min | min | min | min | min | min | min | min |
| Silicone PSA | 0 | 5 | 8 | 13 | 19 | 22 | 25 | 28 |
| Silicone PSA + bioadhesive | 0 | 2 | 4 | 6 | 7 | 4 | 6 | 11 |
| (15% Polyox) | | | | | | | | |
| Silicone PSA + surfactant | 54 | 62 | 62 | 62 | 64 | 63 | 62 | 64 |
| Silicone PSA + surfactant + | 24 | 72 | 86 | 94 | 94 | 96 | 96 | 96 |
| bioadhesive (10% Polyox) | | | | | | | | |
| Silicone PSA + surfactant + | 26 | 72 | 83 | 93 | 93 | 98 | 95 | 96 |
| bioadhesive (2.5% Pemulen) | | | | | | | | |

### Example IV

### Dentifrice Compositions

Dentifrice compositions according to the present invention are shown below with amounts of components in weight %. These compositions are made using conventional methods.

| Components | IVA | IVB | IVC | IVD | IVE | IVF | IVG | IVH | IVJ |
|---|---|---|---|---|---|---|---|---|---|
| Silicone PSA¹ | 5.00 | 3.00 | 2.00 | 1.00 | 3.00 | 10.00 | 3.00 | 5.00 | 3.00 |
| PDMS (100 cSt) | 40.00 | 28.07 | 48.50 | 35.00 | 45.00 | 55.00 | 20.00 | 30.00 | 10.00 |
| Silicone Polyether² | 4.00 | | 3.00 | | 5.00 | 5.00 | 2.00 | 1.00 | 1.00 |
| Pluronic L62 | | 5.00 | | 5.00 | | | | | |
| Sorbitol (70% soln) | | 50.00 | | | 19.83 | 21.86 | 26.26 | | 11.20 |
| Propylene Glycol | | | | 39.20 | | | | | |
| Glycerine | | | | | 10.00 | | | | |
| Pemulen TR-2 | 2.00 | 2.00 | 1.50 | 2.50 | | | 0.30 | | |
| Carbomer 956 | | | | | 2.00 | 3.00 | | 0.50 | 1.00 |
| Urea Hydrogen Peroxide | 8.57 | 2.86 | 5.71 | 8.57 | | | 5.71 | 8.57 | 2.86 |
| Cetylpyridinium Chloride | | | | | 0.53 | | | | |
| Triclosan | | | | | | 0.30 | | | |
| Sodium Fluoride | 0.24 | 0.24 | | 0.24 | 0.24 | 0.24 | 0.24 | 0.24 | 0.24 |
| Stannous Fluoride | | | 0.45 | | | | | | |
| Xanthan Gum | | | 0.47 | | 0.30 | | | 0.45 | |
| Color | 0.30 | 0.20 | 0.20 | 0.30 | 0.20 | 0.20 | 0.20 | 0.20 | 0.20 |
| Flavor | 0.90 | 1.20 | 1.50 | 1.10 | 1.00 | 1.20 | 1.00 | 0.90 | 1.00 |
| Saccharin | 0.30 | 0.45 | 0.70 | 0.50 | 0.65 | 0.70 | 0.40 | 0.70 | 0.50 |
| Na Lauryl Sulfate 28% Soln | | | | | | | 4.00 | 5.00 | 3.00 |
| Ethanol | 34.14 | | 23.31 | | | | | 20.59 | |
| Trisodium Phosphate | 1.45 | 1.40 | 2.00 | 1.50 | 1.75 | 2.00 | 1.40 | 1.75 | 1.40 |
| Monosodium Phosphate | | 0.50 | 0.59 | | 0.50 | 0.50 | 0.40 | | 0.50 |
| Silica Abrasive | | | | | | | 20.00 | 5.00 | 4.00 |
| Mineral Oil/Olive oil | | | | | | | | | 50.00 |
| Sodium stannate | 0.10 | 0.08 | 0.07 | 0.09 | | | 0.09 | 0.10 | 0.10 |
| Water Purified USP | 3.00 | 5.00 | 10.00 | 5.00 | 10.00 | | 15.00 | 20.00 | 10.00 |

¹BIO-PSA ® 7-4202 or 7-4302 amine compatible adhesive resin from Dow Corning.
²Dow Corning DC193 Fluid, Silwet L from General Electric or Silsoft (430, 440, 475, 840) from OSI Specialties

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition for delivering an oral care substance to the oral cavity, comprising:
(a) a silicone pressure sensitive adhesive selected from a silicone/resin copolymer with silicon-bonded hydroxyl radicals, a silicone/resin copolymer with endcapped silicon-bonded hydroxyl radicals and mixtures thereof;
(b) a plasticizing material capable of softening the silicone pressure sensitive adhesive;
(c) a water-soluble bioadhesive polymer;
(d) a hydrophilic surfactant, and
(e) at least one oral care substance.

2. A composition according to claim 1, wherein the silicone/resin copolymer is prepared by polycondensing a silanol endblocked polydialkylsiloxane and a hydroxyl endblocked silicate resin.

3. A composition according to claim l, wherein the endcapped silicone/resin copolymer is prepared by polycondensing a silanol endblocked polydialkylsiloxane and a hydroxyl endblocked silicate resin and further reacting the polycondensation product with a trialkylsilyl endcapping agent.

4. A composition according to claim 3, wherein the silanol endblocked polydiorganosiloxane is silanol endblocked polydimethyl siloxane and the polycondensation product is endcapped using hexamethyldisilazane.

5. A composition according to claim 3, wherein the silicone/resin copolymer is at least about 25% endcapped.

6. A composition according to claim 1, wherein the pressure sensitive adhesive is present in the composition at a level of from about 1% to about 70%.

7. A composition according to claim 1, wherein the plasticizing material is a fluid diorganopolysiloxane polymer comprising repeating units of the formula (R₂SiO)ₙ, where R is selected from the group consisting of monovalent radicals containing from 1 to 6 carbon atoms and terminated by triorganosilyl groups of the formula (R′₃Si) where R′ is a monovalent radical selected from the group consisting of radicals containing from 1 to 6 carbon atoms, hydroxyl groups, alkoxyl groups, and mixtures thereof.

8. A composition according to claim 7, wherein said R radical is selected from the group consisting of methyl, ethyl, propyl, isopropyl, butyl, isobutyl, t-butyl, amyl, hexyl, vinyl, allyl, cyclohexyl, amino alkyl, phenyl, fluoroalkyl and mixtures thereof.

9. A composition according to claim 7, wherein the fluid diorganopolysiloxane polymer plasticizing material is one or a mixture of polydimethylsiloxanes having a viscosity from about 10 to about 1,000,000 centistokes (cSt) at 25° C.

10. A composition according to claim 9, wherein the fluid diorganopolysiloxane polymer plasticizing material is a mixture of a polydimethylsiloxane having a viscosity of from about 10 to about 12,500 cSt and a polydimethylsiloxane having a viscosity ranging from about 12,500 to about 100,000 cSt.

11. A composition according to claim 1 wherein the bioadhesive material is selected from polyhydric alcohols and ether or ester derivatives thereof; polymers or copolymers of ethylene oxide, propylene oxide, acrylate and vinylpyrrolidone; hydroxyethylcellulose polymers; and mixtures thereof.

12. A composition according to claim 1 wherein the hydrophilic surfactant is selected from water soluble nonionic surfactants having a hydrophilic/lipophilic balance (HLB) number ranging from about 3 to about 15.

13. A composition according to claim 12 wherein the hydrophilic surfactant is a silicone polyether.

14. A composition according to claim 1 wherein the oral care substance includes at least one oral care active selected from the group consisting of a teeth whitening active, an anti-tartar agent, a fluoride ion source, an anti-microbial agent, an anti-inflammatory agent, nutrients, an antioxidant, an H2 antagonist, an analgesic active, a desensitizing active, an anti-viral agent, an anti-fungal agent, flavoring agents, sweetening agents, xylitol, opacifiers, coloring agents, chelants, surfactants, pigments, and mixtures thereof.

15. A composition according to claim 14 wherein the oral care substance comprises from about 0.01% to about 50% of the composition.

16. A composition according to claim 14 wherein the oral care substance is a teeth whitening active selected from the group consisting peroxides, metal chlorites, perborates, percarbonates, peroxyacids, persulfates, and mixtures thereof.

17. A composition according to claim 16 wherein the teeth whitening active is selected from the group consisting of hydrogen peroxide, urea peroxide, calcium peroxide, sodium percarbonate, sodium chlorite, potassium chlorite and mixtures thereof.

18. A composition according to claim 16 further comprising a stabilizer for the whitening active, wherein the stabilizer is selected from sodium stannate, sodium acid pyrophosphate, monosodium phosphate and mixtures thereof.

19. A composition according to claim 1 further comprising a solvent selected from the group consisting of hydrocarbon oils, volatile silicones, non-hydrocarbon solvents, and mixtures thereof.

20. A composition according to claim 19, wherein the solvent is selected from the group consisting of ethanol, isododecane, butanone, ethyl acetate, propyl acetate, methyl nonafluoroisobutyl ether, methyl nonafluorobutyl ether and mixtures thereof.

21. A method for controlled and sustained release of oral care actives delivered from a substantially anhydrous silicone adhesive oral care composition by formulating the composition with a mixture of at least one water-soluble bioadhesive material, at least one hydrophilic surfactant, a silicone pressure sensitive adhesive and a plasticizing agent for the silicone adhesive.

22. A method for delivering an oral care substance to at least one surface of the oral cavity, comprising the steps of:
(1) applying a composition to the oral cavity surface(s), wherein the composition comprises:
(a) a silicone pressure sensitive adhesive selected from a silicone/resin copolymer with silicon-bonded hydroxyl radicals, a silicone/resin copolymer with endcapped silicon-bonded hydroxyl radicals and mixtures thereof;
(b) a plasticizing material capable of softening the silicone pressure sensitive adhesive;
(c) a water-soluble bioadhesive polymer;
(d) a hydrophilic surfactant, and
(e) at least one oral care substance, and
(2) allowing the composition to form a film on the surface(s) of the oral cavity.

23. A method according to claim 22 wherein the composition comprises a teeth whitening active and the oral cavity surface to which the composition is applied is the enamel of the teeth.
